(19) United States
(12) Patent Application Publication
(10) Pub. No.: US 2008/0221594 A1
(43) Pub. Date: Sep. 11, 2008

(54) Resorbable truss

(76) Inventors: Ned M. Hamman, Leesburg, IN (US); David A. Werntz, Oak Point, TX (US)

Correspondence Address: HARNESS, DICKEY & PIERCE, P.L.C., P.O. BOX 828, BLOOMFIELD HILLS, MI, 48303, US

(21) Appl. No.: 11/699,441

(22) Filed: Jan. 29, 2007

Related U.S. Application Data
(60) Provisional application No. 60/763,718, filed on Jan. 31, 2006.

(51) Int. Cl. A61B 17/08; A61B 17/56

(52) U.S. Cl. 606/151; 606/76; 606/77

(57) ABSTRACT

A method and apparatus for repairing a bone defect in a cranium includes a frame defining a plurality of openings. The frame may be adapted to generally span the bone defect in an implanted position. Bone-growth promoting material may generally overlap the openings and secured to the frame to cover at least some of the respective plurality of openings. At least one stabilizing member may be attached to the frame for stabilizing the apparatus relative to the cranium.

### CROSS-REFERENCE TO RELATED APPLICATIONS

This application claims the benefit of U.S. Provisional Application No. 60/763,718, filed on Jan. 31, 2006.

### FIELD

The present teachings relate generally to a method and apparatus for repairing a bone defect in a cranium, and more particularly, to a method and apparatus for implanting a resorbable truss assembly.

### BACKGROUND

When performing repair on a cranial defect, it is often necessary to implant material suitable to initiate bone growth. In one example, demineralized bone may be utilized at the defect site. However, in some instances, such material on its own may not be able to hold the desired shape of the skull without underlying soft tissue. In some cases where a cranial defect is large, it may be especially challenging to successfully place such bone growth material at the repair site while maintaining conformance to the curvature of the skull. In certain instances, it may be necessary to utilize supplemental external orthotics to assist proper location of the bone growth material.

### SUMMARY

A method and apparatus for repairing a bone defect in a cranium includes a frame defining a plurality of openings. The frame may be adapted to generally span the bone defect in an implanted position. Bone-growth promoting material may generally overlap the openings and secured to the frame to cover at least some of the respective plurality of openings. At least one stabilizing member may be attached to the frame for stabilizing the apparatus relative to the cranium.

The frame may be formed of resorbable material. The resorbable material may be Lactosorb®, polylactic acid material, polyglycolic acid material, caprolactone or others. The frame may define an arcuate contour in an implanted position. The bone-growth promoting material may be osteoconductive material (OC) material, osteoinductive (OI) material, osteogenetic (OG) material or other materials adapted to promote bone growth. The bone-growth promoting material may be attached to the frame by any suitable method such as suturing for example.

The plurality of openings may define a series of repetitive geometric shapes. The bone-growth promoting material may define shapes complementary to the series of repetitive shapes. In one example, the repetitive geometric shapes include triangles.

Further areas of applicability of the present teachings will become apparent from the detailed description provided hereinafter. It should be understood that the detailed description and specific examples are intended for purposes of illustration only and are not intended to limit the scope of the claims.

### BRIEF DESCRIPTION OF THE FIGURES

The present teachings will become more fully understood from the detailed description and the accompanying drawings, wherein:

FIG. 1 is a side perspective view of an exemplary bone defect in a cranium;

FIG. 2 is a partial perspective view of an exemplary resorbable truss according to the present teachings;

FIG. 3 is a plan view of an exemplary resorbable truss template according to additional features;

FIGS. 4A-4C illustrate an exemplary sequence for preparing a plurality of sheets of bone-growth promoting material;

FIG. 5 is a detail view of the truss shown in FIG. 2 taken at detail A illustrating some of the sheets of material in FIG. 3 being attached to the truss according to the present teachings; and

FIG. 6 is a side perspective view of the cranium of FIG. 1 shown with the assembled truss implanted at the defect according to an exemplary method.

### DETAILED DESCRIPTION

The following description is merely exemplary in nature and is in no way intended to limit the application or uses. Those skilled in the art will appreciate that the following teachings can be used in a much wider variety of applications than the examples specifically mentioned herein. More specifically, while the following discussion is specifically directed toward an apparatus and method for repairing a defect in a cranium, the same may be applied to other bone defects in the human anatomy.

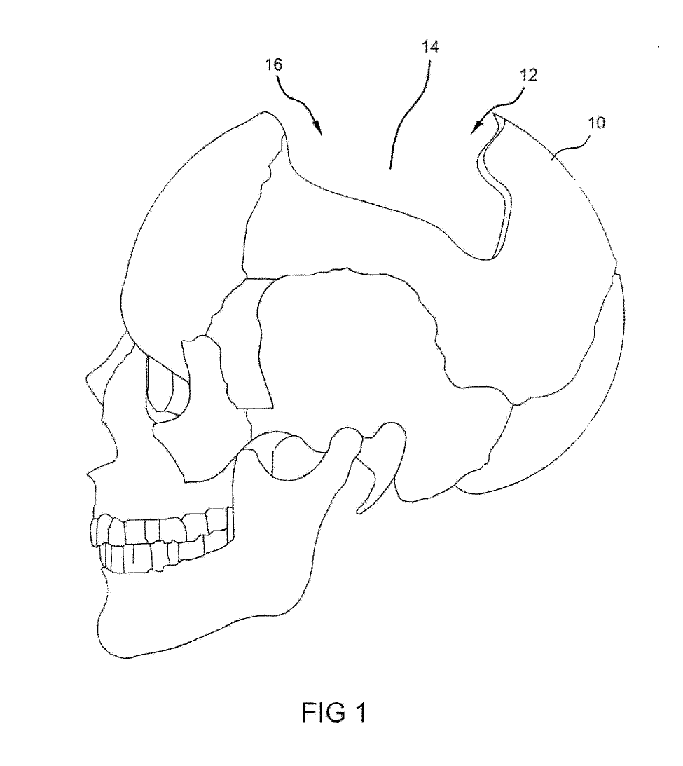

With initial reference to FIG. 1, a cranium 10 having a large defect 12 is shown. The large defect 12 is generally defined by surrounding bone outlining an opening 14 in the cranium 10. As used herein, the opening 14 and the area of bone generally surrounding the large defect 12 will be referred to collectively as a defect site 16. As will be appreciated, the defect 12 illustrated in FIG. 1 is merely exemplary and the method for implanting the resorbable truss assembly according to the present teachings may be adapted for implantation at a cranial defect site having various shapes.

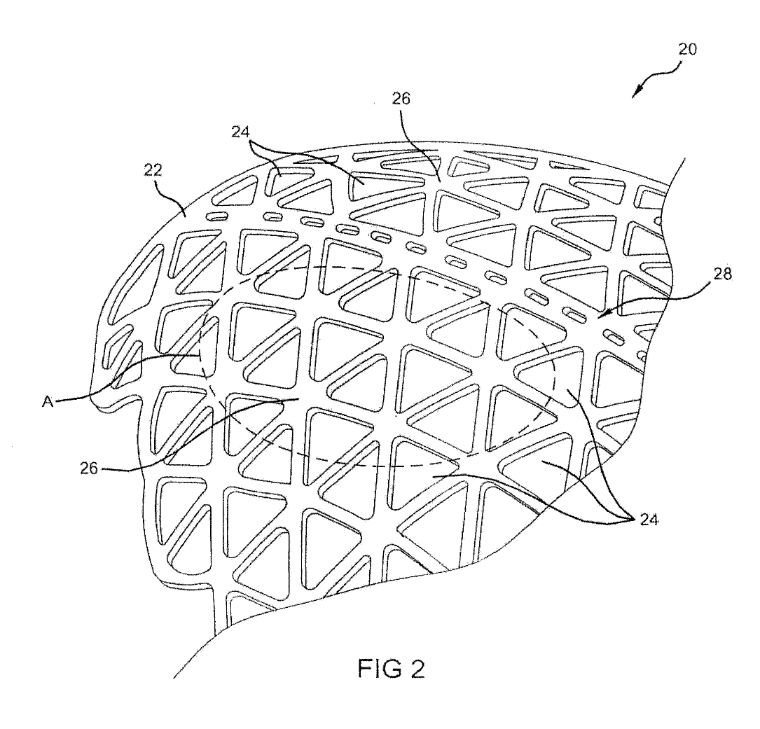

Turning now to FIG. 2, a partial detail view of a resorbable truss 20 according to the present teachings is shown. The resorbable truss 20 may be formed of any suitable resorbable material such as, but not limited to, LactoSorb® distributed by Biomet of Warsaw, Ind., polylactic acid (PLA) materials, polyglycolic acid (PGA) materials, caprolactone, or any other resorbable polymer, co-polymer, or terpolymer. As shown, the exemplary truss 20 generally defines a frame 22 having a plurality of passages or openings 24 formed therein. The frame 22 may be formed as a planar sheet and subsequently shaped to the contour of a patient's cranium. In one example, the frame 22 may be formed by way of a molding process. Other methods may be utilized. For instance, the frame 22 may be custom manufactured utilizing geometry according to the specific measurements of the patients' anatomy.

While the openings 24 are specifically illustrated as triangular in shape, the openings 24 may be defined by any geometric shape such as circular, rectangular, diamond, octagonal and others. The openings 24 may be formed across the frame in a regular or irregular fashion. Alternatively, a combination of openings defining distinct shapes may be used. Truss junctions 26 are identified at locations where the truss 20 transitions between adjacent openings 24. In one example, a transition region 28 may be defined across a portion of the truss 20. As will be described below, the truss 20 provides a structural framework to maintain geometry of the truss assembly as a whole when implanted.

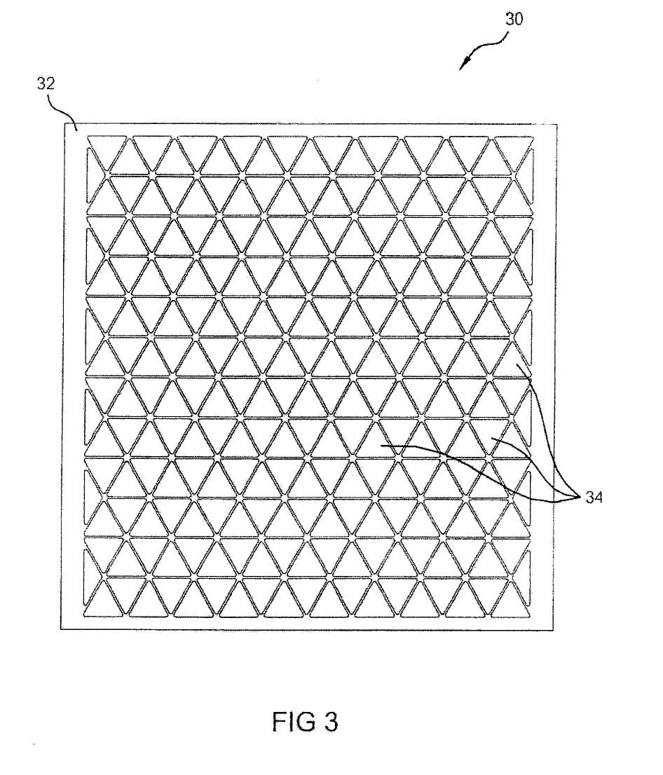

Turning now to FIG. 3, an exemplary truss template 30 is shown. The truss template 30 may be preformed in a generic sheet 32 such as a rectangular sheet and subsequently shaped or cut to conform to a specific application. As illustrated, the truss template 30 provides a repetitive pattern of openings 34 in the shape of triangles. It is appreciated that other geometric shapes may be used. In one example, the openings 34 may define an average width of between 4 mm and 40 mm. The truss 30 may subsequently be cut or trimmed to a desired footprint. In this way, the truss 30 may be cut to a specific size to correspond to a given patients' anatomy, such as for example the defect site 16 (FIG. 1). Once cut to a desired footprint, the truss template 30 may be shaped into a spherical geometry to conform to a patient's cranium. In one example, the truss template 30 is pliable and may be shaped manually by hand. The material properties of the resorbable truss template 30 allow a surgeon to shape the truss as needed and further allow the truss 30 to substantially maintain its geometry once shaped.

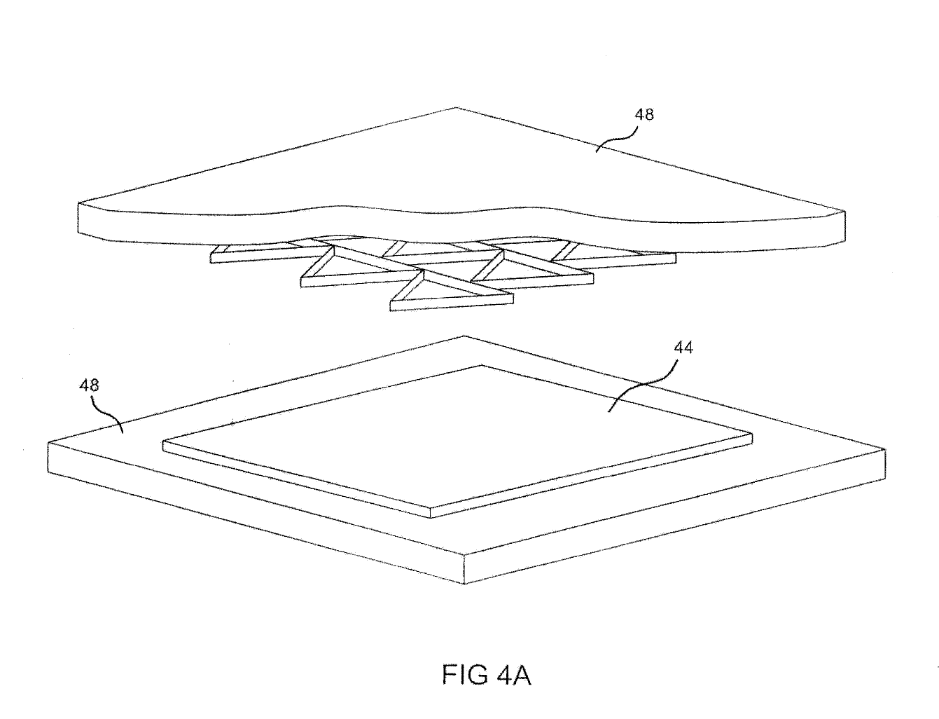

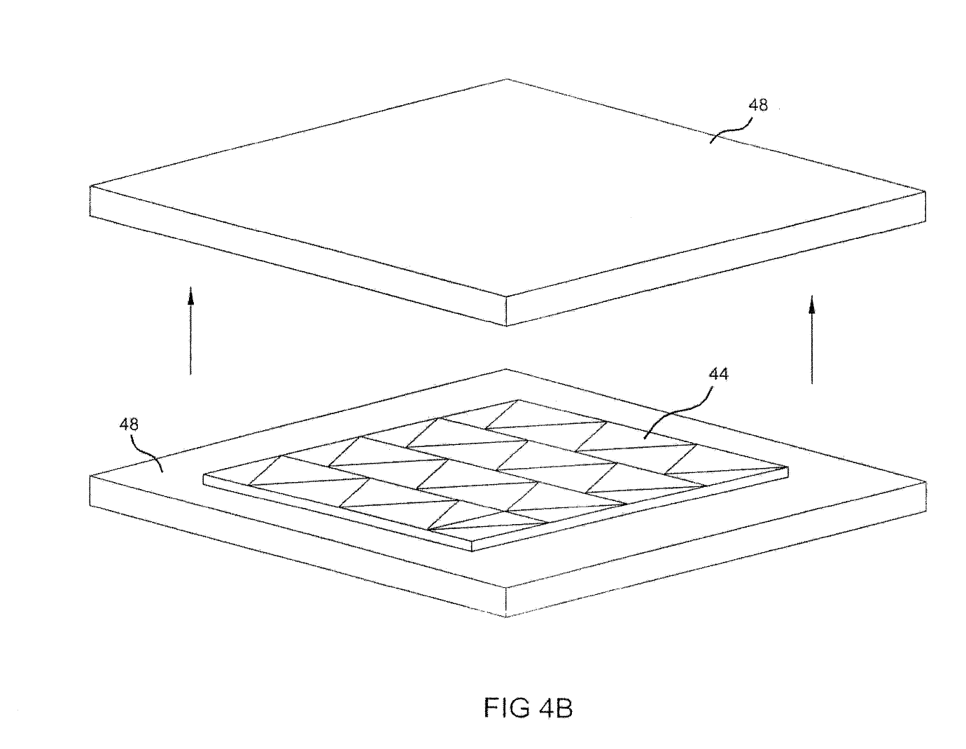

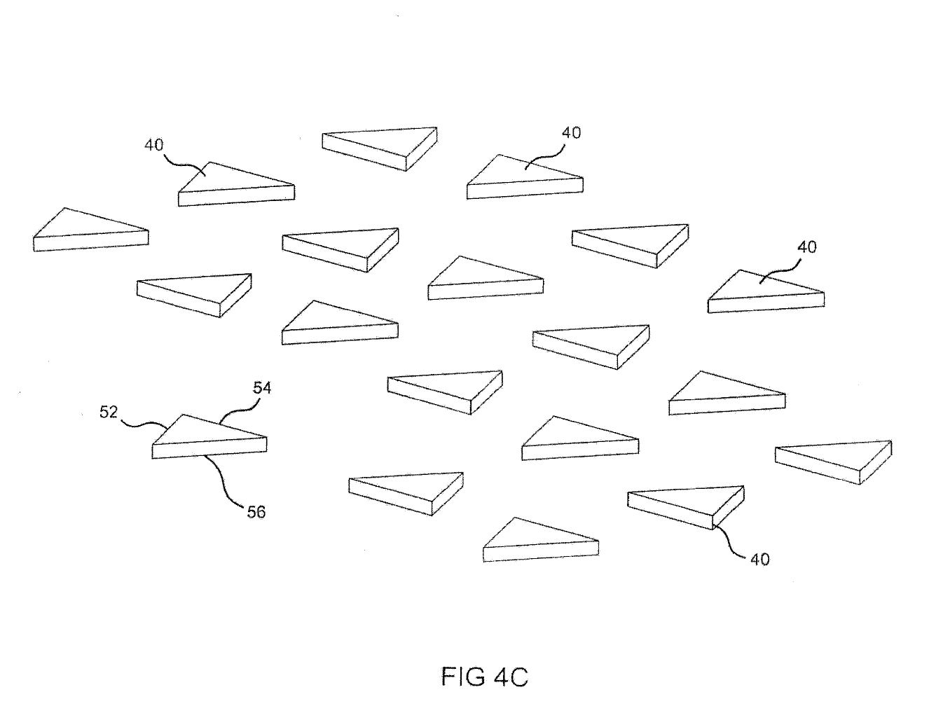

With continued reference to FIGS. 2 and 3 and further reference to FIGS. 4A-4C, sheets of bone-growth promoting material 40 and an exemplary method of assembling the sheets of bone-growth promoting material 40 to the truss 20 will be described. In general, the plurality of sheets of bone-growth promoting material 40 may be attached to the truss 20 and adapted to collectively cover at least some of the respective plurality of passages 24. In one example, the sheets of bone-growth promoting material 40 may generally define a geometry complementary to the respective openings 24 in the truss 20.

With specific reference to FIGS. 4A-4C, in one example, the plurality of sheets of bone-growth promoting material 40 may be formed from a larger template of material 44 (FIG. 4A). In this way, a press or other cutting mechanism 48 may be used to cut the template of material 44 or a portion of the template of material 44 into the plurality of sheets 40 as needed (FIG. 4B). According to the example shown in FIG. 4C, the sheets 40 each define a triangular shape having first second and third sides 52, 54, and 56, respectively.

As described, the sheets 40 are formed of a material adapted to initiate bone growth. The sheets 40 may be formed of at least one of an osteoconductive (OC) material, an osteoinductive (OI) material and an osteogenic (OG) material. The OC material may be an hydroxyapatite (HA) scaffold. For example, Calcigen™ PSI (Porous Synthetic Implant), ProOsteon®, both distributed by Biomet of Warsaw, Ind., a collagen sheet, a gel foam or other material allowing bone ingrowth. The OI material may include a demineralized bone matrix (DBM) strip, DBM putty, Lyophilized DBM, bone morphogenic proteins (BMP's), growth factors including a platelet derived growth factor (PDGF), a vascular endothelial growth factor (VEGF), platelet concentrate or others may be used or may be added to the OC material. The OG material may be autogenous cells (such as stem cells derived from bone marrow aspirate or autologous fat), an autogenous bone graft such as a rib graft, fibular strut, or split-thickness calvarial graft. Other materials are contemplated.

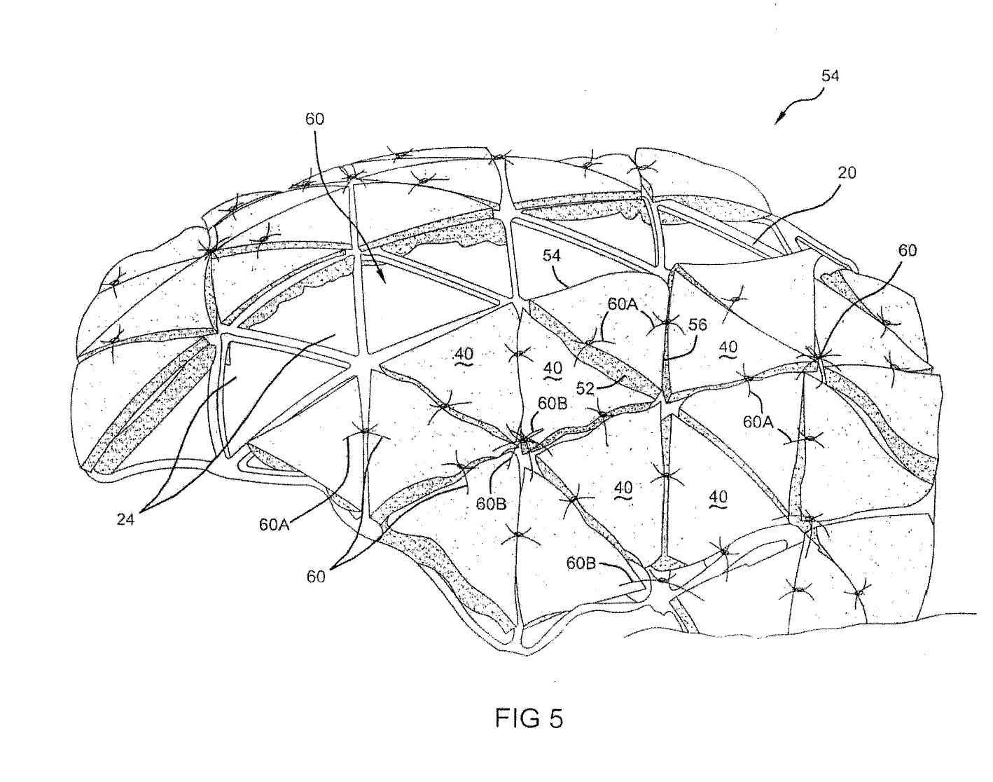

As illustrated in FIG. 5, a series of sheets 40 are shown attached to a respective series of openings 24 in the truss 20 to form a truss assembly 54. In one example, sutures 60 may be used to attach the sheets 40 to the truss 20. The sutures 60 may be resorbable. In one method, sutures 60A may be used around respective midpoints of the sides 52, 54, and 56 of the sheets. In another method, sutures 60B may be alternatively, or additionally used at respective corners of the sheets 40 wherein the sutures 60B wrap around respective truss junctions 26. Other fastening techniques may be employed for coupling the sheets 40 to the truss 20. Portions of the truss 20 may become partially deformed during suturing and/or the shaping process described above. For illustrative purposes, a row 60 of the truss 20 is shown prior to having sheets 40 attached.

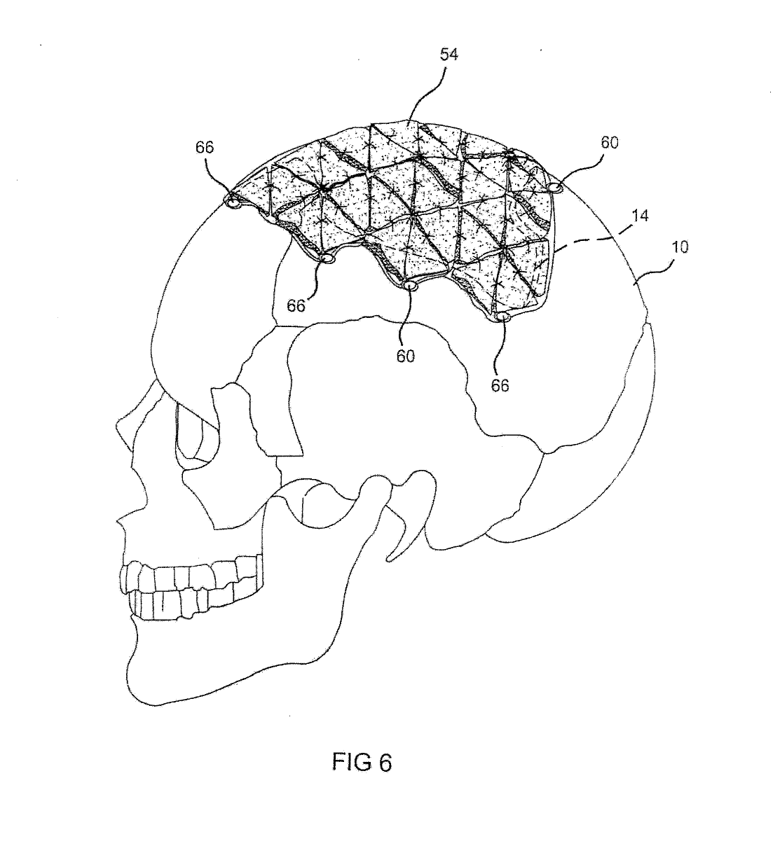

With reference now to FIG. 6, once the respective sheets 40 are suitably attached to the truss 20, the truss assembly 54 may be implanted at the defect site 16. In one example, bone screws 66 may be utilized at random or specified locations around the perimeter of the truss assembly 54 to attach the truss assembly 54 to the cranium. In one example, bone screws 60 may be utilized at or near respective truss junctions 26. Additionally or alternatively, the bone screws 60 may be used elsewhere on the truss assembly 54. Other means of fixation may be employed. While the truss assembly 54 is shown partially overlapping the surrounding bone outlining the opening 14 in the cranium 10, the truss assembly 54 may be configured to span a greater, or reduced distance across the opening 14. Once implanted, the truss 20 of the truss assembly 54 provides a framework for structural integrity at the repair site as a whole. Meanwhile, the sheets 40 facilitate healing across the defect site 16 by initiating bone growth. As the defect site 16 heals, new bone grows over the opening 14 as the truss 20 sheets 40 and sutures 60 are all resorbed into the body.

Those skilled in the art can now appreciate from the foregoing description that the broad teachings can be implemented in a variety of forms. Therefore, while this description includes particular examples, the teachings should not be so limited because other modifications will become apparent to the skilled practitioner upon a study of the drawings, the specification and the following claims.

What is claimed is:

1. An apparatus for repairing a bone defect in a cranium comprising:
a frame defining a plurality of openings and adapted to generally span the bone defect in an implanted position;
bone-growth promoting material generally overlapping the openings and secured to the frame to cover at least some of the respective plurality of openings.

2. The apparatus of claim 1, further comprising a stabilizing member attached to the frame and adapted to stabilize the apparatus relative to the cranium.

3. The apparatus of claim 1 wherein the frame is formed of resorbable material.

4. The apparatus of claim 3 wherein the resorbable material is selected from the group consisting of Lactosorb, polylactic acid material, polyglycolic acid material and caprolactone.

5. The apparatus of claim 3 wherein the frame defines an arcuate contour in the implanted position.

6. The apparatus of claim 5 wherein the frame is pliable.

7. The apparatus of claim 3 wherein the plurality of openings are formed in a series of repetitive geometric shapes and wherein the material defines a section of material having a shape complementary to the series of repetitive geometric shapes.

8. The apparatus of claim 7 wherein the repetitive geometric shapes comprise triangles.

9. The apparatus of claim 7 wherein the section of material is attached to the frame by sutures.

10. The apparatus of claim 9 wherein the bone-growth promoting material are selected from the group consisting of an osteoconductive material (OC) material, an osteoinductive (OI) material, and an osteogenetic (OG) material.

11. The apparatus of claim 10 wherein the OC material includes at least one of a hydroxyapatite (HA) scaffold and a collagen sheet.

12. The apparatus of claim 10 wherein the OI material includes at least one of a demineralized bone matrix (DBM) strip, a DBM putty, a lyophilized DBM, bone morphogenic proteins (BMP), platelet derived growth factor (PDGF), vascular endothelial growth factor (VEGF) and platelet concentrate.

13. The apparatus of claim 10 wherein the OG material includes at least one of autogenous cells and an autogenous bone graft.

14. The apparatus of claim 13 wherein the autogenous cells comprise stem cells derived from at least one of bone marrow aspirate and autologous fat.

15. The apparatus of claim 13 wherein the autogenous bone graft comprise at least one of a rib graft, a fibular strut and a split-thickness calvarial graft.

16. The apparatus of claim 1 wherein the stabilizing member includes a bone screw.

17. An apparatus for repairing a bone defect in a cranium comprising:
a frame defining a plurality of openings, the frame adapted to generally span the bone defect in an implanted position;
a plurality of sections of bone-growth promoting material adapted to collectively cover at least some of the respective plurality of openings; and
a plurality of fastening members operably coupling the plurality of sections of bone-growth promoting material to the frame.

18. The apparatus of claim 17 wherein the plurality of sections of bone growth promoting material each define a geometry complementary to the plurality of openings.

19. The apparatus of claim 17 wherein the frame is formed of resorbable material.

20. The apparatus of claim 19 wherein the resorbable material is selected from the group consisting of Lactosorb, polylactic acid material, polyglycolic acid material and caprolactone.

21. The apparatus of claim 17 wherein the sections of bone-growth promoting material are selected from the group consisting of an osteoconductive material (OC) material, an osteoinductive (OI) material, and an osteogenetic (OG) material.

22. A method comprising:
securing a section of bone-growth promoting material to a frame having a plurality of openings; and
securing the frame to the cranium in a location generally spanning a bone defect in a cranium.

23. The method of claim 22 wherein the frame includes resorbable material.

24. The method of claim 22 wherein securing the section of bone-growth promoting material includes covering at least some of the plurality of openings.

25. The method of claim 22 wherein securing the section of bone-growth promoting material includes suturing the section of bone-growth promoting material to the frame.
